(19) United States
(12) Patent Application Publication
(10) Pub. No.: US 2006/0195051 A1
(43) Pub. Date: Aug. 31, 2006

(54) Posture monitoring device and method of use thereof

(76) Inventors: Elma O. Schnapp, Memphis, TN (US); Moacir Schnapp, Memphis, TN (US); Eric C. Schnapp, Memphis, TN (US); William D. Schnapp, Memphis, TN (US); Denise E. Schnapp, Memphis, TN (US)

Correspondence Address: H. Roy Berkenstock;Wyatt, Tarrant & Combs, LLP, Suite 800, 1715 Aaron Brenner Drive, Memphis, TN, 38120-4367, US

(21) Appl. No.: 11/066,505

(22) Filed: Feb. 25, 2005

(51) Int. Cl. A61B 5/103

(52) U.S. Cl. 600/595

(57) ABSTRACT

The present invention discloses a posture monitoring device. The device includes a tilt sensor, an alarm, and a recording device to track the changes in posture. The device has been designed to overcome the currently existing problems associated with other devices that detect poor posture. The device also includes the ability to modify the target posture angle and the amount of time during which poor posture is detected prior to activation of the alarm.

### GOVERNMENT SUPPORT CLAUSE

Not applicable.

### FIELD OF THE INVENTION

The present invention relates generally to the field of posture management. Specifically, the present invention relates to a device which identifies incorrect posture and signals for the correction of the posture.

### BACKGROUND OF THE INVENTION

The health and comfort of humans are enhanced by maintaining proper posture while standing, sitting, etc. Medical reports have clearly shown that maintaining correct posture results in fewer injuries. Specifically, with regard to elderly women, correct posture during the medical therapy for osteoporosis is a necessity. As females age, they may be susceptible to calcium loss, resulting in osteoporosis. Although medications are available to help restore the calcium and increase bone strength, unless the patient's posture is corrected during therapy, it is possible that the rounded shoulders and slumping posture, often associated with osteoporosis, will remain after treatment. Further, proper posture prevents curvature of the spine, such as kyphosis and scoliosis, even for individuals not subjected to osteoporosis treatment.

Other attempts have been made to provide a device which alerts a user to correct their posture. However, each of the previous attempts is flawed with regard to the correction of the posture and/or the ease of use. Often times, the previous attempts have resulted in cumbersome devices, which are difficult to use. Thus, it is an object of the present invention to provide a posture alert device which may be worn inconspicuously and effectively alert a user when to correct their posture.

### SUMMARY OF THE INVENTION

The present invention discloses a posture alert device for alerting a user of their poor posture. The device provides the advantage of accurately measuring the tilt angle of the user's posture while minimizing the number of false alarms received by the user for alleged poor posture. Additionally, as the user transitions from, for example, a standing position to a seated position, the device allows the user to easily reset it so that the user's posture while seated, etc., is accurately monitored. Also, the device makes record of the user's posture swings so that such information may subsequently be analyzed. More specifically, the device includes a housing which surrounds the device, a first circuit board attached to the housing, a second circuit board attached to the first circuit board, wherein the second circuit board is attached to the first circuit board at about a right angle, a tilt sensor attached to the second circuit board and an alarm attached to the first circuit board.

In certain embodiments, the device further includes a switch attached to the first circuit board, wherein the switch is operationally connected to the tilt sensor so that the switch resets the tilt sensor. Certain embodiments of the device may have a clip, or attachment loop, attached to the housing so that the device attaches to a user. In alternate embodiments of the device, the alarm may be a vibrator unit, a light, or a speaker. In certain embodiments, the housing has a thickness of from about 0.010 inch to about 0.070 inch.

In still other embodiments, the device further includes a timing sensor and a monitoring circuit for monitoring the maximum swings of the spine angle. Other embodiments may include a recorder, also called a memory storage device, for recording the maximum swings of the spine angle. Still other embodiments include a memory storage device for recording and playing back the recorded angles. In certain embodiments, the angle detecting sensor may be digital. In other embodiments the housing may have a length of up to 1.35 inches and a width of up to 1.10 inches.

Accordingly, one object of the present invention is to provide a device for conveniently and accurately monitoring the posture of a user.

Another object of the present invention is to provide a device that does not require uncomfortable fixation to the torso of the user.

Still another object of the present invention is to provide a device that provides flexible settings so that the user determines the type of alarm notification and the amount of time allowed for a posture violation before alarm notification.

Another object of the present invention is to provide a small device which may be quickly and conveniently removed when a user wants to suddenly stop monitoring his or her posture.

### BRIEF DESCRIPTION OF THE DRAWINGS

FIG. 1A shows a cross section of an elevated side view a first embodiment of the present invention.

FIG. 1B is a front view of a first embodiment of the posture monitoring system showing the reset button.

FIG. 1C is a perspective view of the posture monitoring system, without the housing, showing the positions of the components attached to the first circuit board.

FIG. 2A is a cross section of an elevated side view of a first embodiment of the present invention, showing the alarm and battery which are located underneath the first circuit board.

FIG. 2B is a top view of the first embodiment of the posture monitoring system. FIG. 2B shows the components attached to the top side of the first circuit board and the components attached to the underside are shown in phantom lines.

FIG. 3A is a front view of a second embodiment of the present invention with dashed lines showing the wall thickness of the housing and phantom lines showing the positioning of the alarm, second circuit board, and tilt sensor on the underside of the first circuit board.

FIG. 3B is a cross section of an elevated side view of a second embodiment of the present invention.

FIG. 4 is a flow chart showing the steps of selecting the operating mode of the present invention.

FIG. 5 is a flow chart showing the steps of using the present invention.

FIG. 6 is a schematic drawing showing the operational connections of the components of the present invention.

### DESCRIPTION OF THE PREFERRED EMBODIMENTS

The present invention is a posture monitoring system 10, also called a posture alert device, which includes a housing 12, a timing sensor in the form of a microprocessor 42, a tilt sensor 16, also called an angle detecting sensor, an alarm 18, and monitoring circuit hardware that includes a memory storage device 44. The posture monitoring system 10 provides an alert, or notice, to a user when the user's posture is in violation of the specific settings of the system 10. As further described herein, the system 10 may be set to specific ranges for tilt, and the amount of time during which a user may be in violation of a specific tilt range.

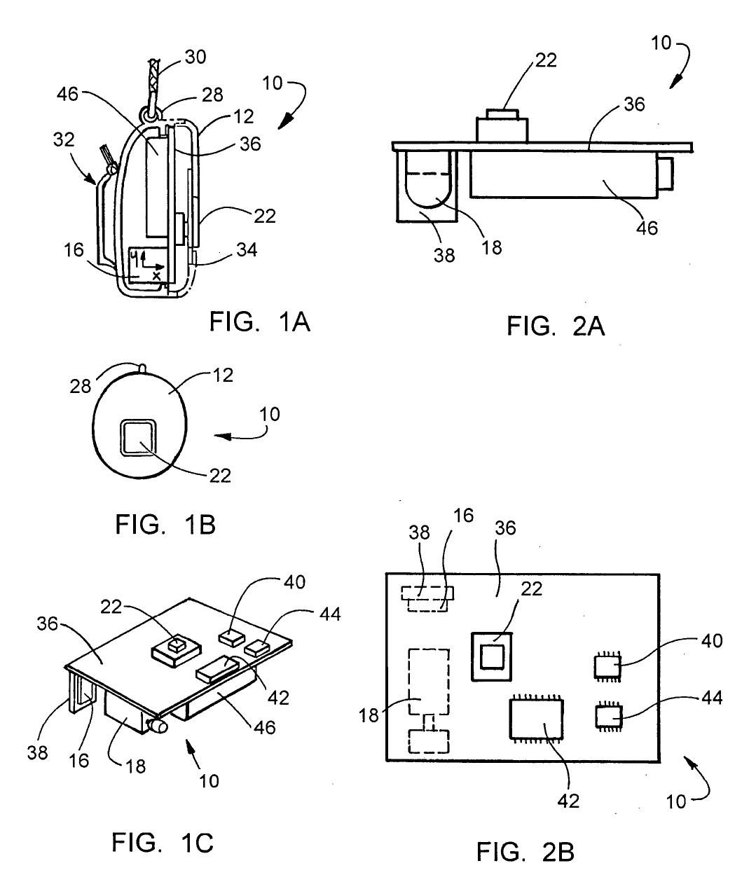

Shown in FIGS. 1A-1C is a first embodiment of the present invention. FIG. 1B shows a front view of the invention having a cylindrical shape. FIG. 1A, which is a cross sectional view, shows the housing 12, the tilt sensor 16, the reset button 22, the first circuit board 36, the elastomeric membrane 34 covering the reset button 22, the clip 32, the battery 46, and the attachment loop 28 attached to a necklace 30. FIG. 1C is a perspective view of the system 10 without the housing 12. There is shown the second circuit board 38, which is attached to the first circuit board 36 at approximately a right angle, the voltage regulator 40, the microprocessor 42, the memory storage device 44, the battery 46, the alarm 18, the tilt sensor 16, and the reset button 22. Shown in FIGS. 1A, 1C, 2A, and 2B is the length of the system 10. The value of the length may be up to 3.5 inches. In certain embodiments, the length is 3.17 inches. In alternate embodiments, the length is up to 1.35 inches.

FIGS. 2A and 2B show the invention without the housing 12 so that the interior components may be easily seen. Shown in FIG. 2A is a side view of the system 10. Shown in FIG. 2B is a view of the top of the first circuit board 36. Certain components located on the underside of the first circuit board 36 are shown with phantom lines. In a second embodiment, the system 10 may have a length of up to 0.75 inches and a width of up to 1.75 inches. In certain embodiments of the present invention, the length of the invention is 0.59 inches and the width is 1.5 inches. Thus, as seen in FIGS. 1 and 2, the system 10 is contained within an extremely small space, such as a length of 3.17 inches and a width of 0.67 inches, or a length of 0.59 inches and a width of 1.50 inches. In certain embodiments of the present invention, the size of the invention is approximately 1.5 inches by 1 inch by 0.35 inches, which includes an injection molded housing 12. Furthermore, the system 10 is unitary and does not result in the need to affix various structures to different parts of the user's body.

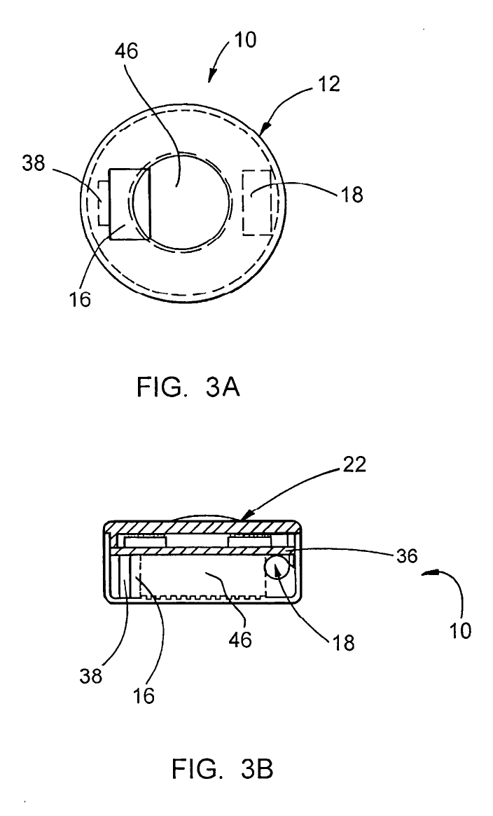

Shown in FIGS. 3A and 3B is another embodiment of the present invention. In FIG. 3A is shown a cross section from the front view of the device 10. There is shown the housing 12, alarm 18, battery 46, second circuit board 38, and tilt sensor 16. FIG. 3B is a cross section of an elevated side view and shows the same components, in addition to the first circuit board 36 and reset button 22.

The system 10 may be constructed as disclosed herein. The housing 12 may be constructed of any rigid material, which is sufficient to provide a point of attachment for the other components of the invention disclosed herein. By way of illustration, and not limitation, examples of materials which may be used for the housing 12 include metal, plastic, rubber, glass, or ceramic material. In certain embodiments, the housing 12 wall may have a thickness of approximately 0.010 inches to approximately 0.070 inches, depending upon the material of construction.

Certain components of the present invention may be commercially available. The timing sensor which includes the microprocessor 42 is commercially available as model PIC 16C57-20/SS from Microchip Technology, Inc., 2355 West Chandler Blvd., Chandler, Ariz. 85224. The type of microprocessor that is suitable for the instant invention has low power consumption, low voltage requirement (3.3 volts preferred, and able to operate at less than six volts), has a “sleep” mode, has the ability to measure the ON and OFF times of a digital input to within approximately 100 microseconds, has basic math function including exponents and trigonometry, a minimum of three digital inputs and two digital outputs, and an overall small size to fit within the specifications disclosed herein. Accordingly, alternate microprocessors may be used.

The tilt sensor 16 is commercially available, for example, from a source such as Parallax, Inc., 599 Menlo Drive, Suite 100 Rocklin, Calif., 95765. The alarm 18 may be a device which notifies the user when a violation of posture occurs. Accordingly, the alarm may be a vibrator motor to provide a motion signal, a light to provide a vision signal, or a speaker to provide an audible signal. In certain embodiments, the alarm 18 is a vibrator motor, which is commercially available, for example, from Jinlong Machinery and Electronics, Inc., Baixiang Industry Zone, Yeuging, Wenzhou, Zhejiang, China, P.E. 325603. Other sources for a vibrator unit are well known in the art.

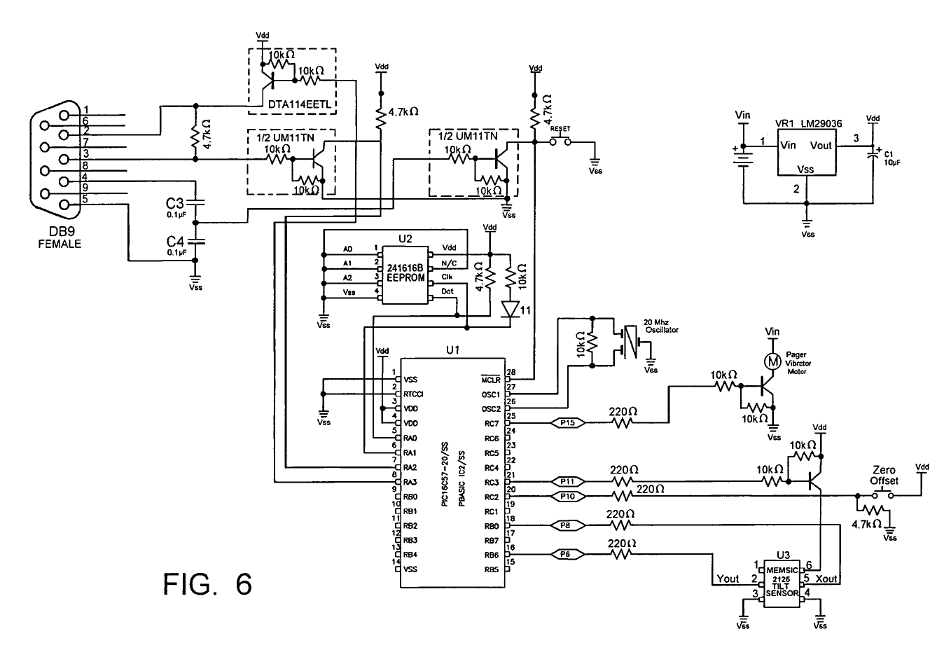

Finally, the monitoring circuit, which includes a memory storage device 44, monitors and stores information obtained by the microprocessor 42 and the tilt sensor 16. Stated another way, the monitoring circuit hardware consists of three major components: a tilt sensor 16, a microprocessor 42 for acquiring data from the tilt sensor 16, performing calculations on the data, and initiating control outputs to the alarm 18 and battery 46, and a memory storage device 44. A memory storage device 44, such as the 24LC16B EEPROM is commercially available, for example, from Microchip Technology, Inc., 2355 West Chandler Blvd., Chandler, Ariz. 85224. A tilt sensor 16, such as model 2125 of Memsic, Inc., 800 Turnpike Street, Suite 202, North Andover, Mass. 01845, is commercially available. Information regarding the type of microprocessor 42 is provided above. Each of the components listed in this paragraph are readily commercially available and are individual devices which are well known in the industry. Within the relevant art, one of ordinary skill in the art knows how to operationally connect the components described herein so that the present invention performs the stated functions. Stated another way, operationally attaching the parts of this invention is accomplished by use well known methods which are known to those of ordinary skill in the art. By way of illustration, and not limitation, FIG. 6 provides a schematic drawing of the operational connections of the present invention. Furthermore, appropriately sized parts may be obtained from the listed commercial sources in order to construct an embodiment of the system 10, having the specific length and width characteristics as further described herein.

In certain embodiments of the present invention, the system 10 is capable of storing data for evaluation purposes. Accordingly, in order to facilitate the transfer of data, it is necessary that the system include an RS-232 or USB connection and EEPROM or FLASH RAM MEMORY. For example, each time an alert occurs, the recorded data may include the time, date, and tilt angle which resulted in the violation. Accordingly, the recorded data may be stored as a comma delimited file so that it may be transferred via a USB connection, or the like, into a spreadsheet, or the like. The recorded data may include a time stamp and the reference tilt angle. When the recorded data is displayed, on a monitor or in printed form, the data may be analyzed for medical diagnostic purposes. Alternatively, certain embodiments of the present invention may have wireless data transmission via blue tooth or infrared transmitters or other appropriate wireless transmission. With regard to the power source, the invention may include a replaceable battery source or a rechargeable battery source. The manner of transferring data through the described avenues is well known in the art and is easily accomplished by one of ordinary skill in the art.

With regard to the tilt sensor 16 of the present invention, the tilt sensor 16 measures both the x and y axes. While it is not necessary to measure both x and y axes, it only takes two inputs on a microcontroller to do so. By measuring both the x and y axes, an overall resolution of the tilt and orientations is provided.

Having disclosed how to make the system 10, information is provided with regard to using the system 10. A user may attach the device 10 to an article of clothing such as a shirt or under garment by use of the clip 32 or attachment loop 28, as seen in FIGS. 1A and 1B. The present invention may also include other mechanisms of attachment such as a pendant or broach pin, or button-back pin. In certain embodiments, when the system 10 is attached to an article of clothing adjacent the sternum, such as when used as a broach pin, the user may set the alarm to vibrate so that they are discretely notified when a posture violation occurs. In such a situation, the user easily feels the vibration on their sternum yet, little audible sound will occur. As further described below, the user sets two variables, the allowable posture angle and the amount of time allowed during a posture violation before the alarm activates. Then the user presses a reset button 22 to establish a reference tilt angle having a zero degree tilt. The angle measured by the tilt sensor 16 may be monitored ever 10 seconds, based upon the user's preferences. When the tilt sensor 16 measures a tilt angle greater than the preset posture tilt angle, then the user receives notification by way of the alarm 18. In certain embodiments, the alarm 18 may be signaled as a pulse of approximately one second of a vibrator motor. In certain embodiments of the present invention, if, after a ten-second period, the user's posture continues to be in violation, the vibrator motor will then again pulse for one second. This will continue in ten-second intervals until the user maneuvers his or her body so that the tilt angle of the device is within the preset range of the system 10.

In certain embodiments of the present invention, if the measured tilt angle exceeds that which has been preset, the system 10 will began sampling the tilt angle at half-second intervals and will calculate a ten-second moving average of the tilt angle. If the ten-second average tilt-angle exceeds the preset alert tilt angle, then the alarm 18 will activate.

In other embodiments of the present invention, if no movement is detected for a five-minute period, then the device is put into a “sleep” mode and the device will evaluate the tilt angle at a frequency of five-minute intervals. In still other embodiments of the present invention, if movement has occurred or if the reset button 22 is pushed, then the system 10 will resume monitoring at 10-second intervals.

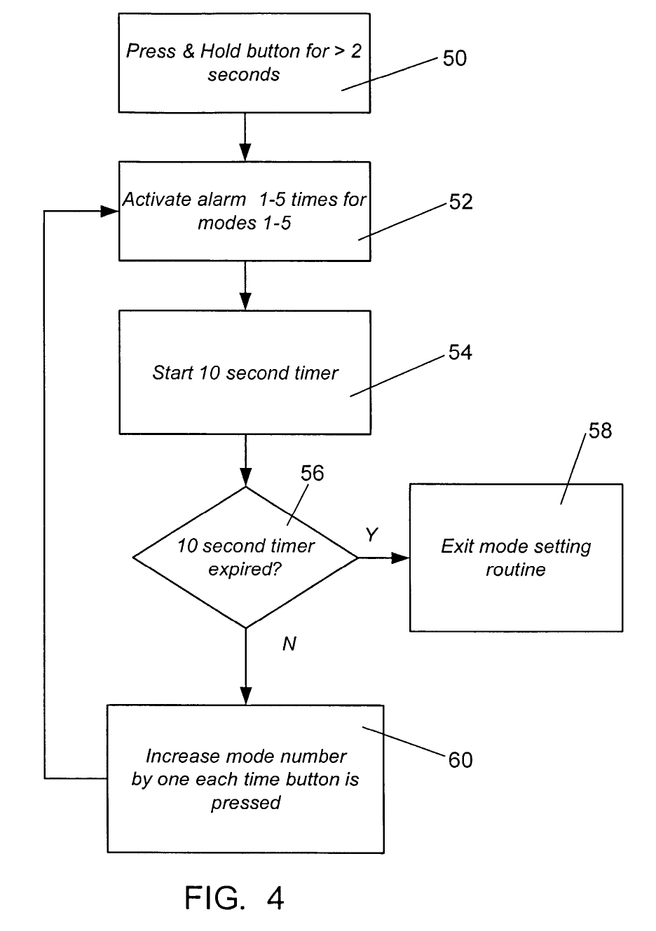

In order to use the system 10 as described above, it is first necessary to provide certain settings so that the settings so that the system 10 may operate as intended. As further described herein, and as shown in the flow-chart of FIG. 4, the system 10 may be programmed to alert a user when their posture results in a tilt angle of from 5 to 30 degrees, or greater. In certain embodiments, the tilt angle may be indicated in five-degree increments. In certain embodiments of the present invention, the system 10 has a plurality of selectable operating modes. For example, the system 10 may have five user-selectable operating modes. The operating modes may be accessed by pressing a reset button 22 and holding it for approximately 3 seconds. Upon accessing the operating mode the alarm 18 will activate one to five times to indicate the current operating mode. The mode can be changed by pressing the reset button 22 again within 10 seconds. If the reset button 22 is pressed again within 10 seconds, the operating mode will increase by one each time the reset button 22 is pushed. After the system 10 is in operating mode 5, pressing the reset button 22 again will result in the operating mode cycling back to mode number 1. In certain embodiments, preset angles are associated with each of the operating modes. Accordingly, in certain embodiments, operating mode number 1 sets the tilt alert angle to 10 degrees. Operating mode number 2 sets the tilt alert angle to 15 degrees. Operating mode number 3 sets the tilt alert angle to 20 degrees. Operating mode 4 sets the tilt alert angle to 25 degrees. Operating mode number 5 sets the tilt angle to 30 degrees. If the ten-second time out expires, the device will automatically exit the set-up procedure and return to normal operation.

Referring specifically to FIG. 4, there is shown a flow chart providing a method of selecting the operation mode of a certain embodiment of the present invention. First, a user presses and holds the reset button 22 for more than two seconds 50. Then, the alarm 18 activates from one to five times 52, which represents the operating mode, one to five, for which the device 10 is set. A ten second timer is started 54. In certain embodiments, mode one is a tilt angle of 10 degrees, mode two is a tilt angle of 15 degrees, mode three is a tilt angle of 20 degrees, mode four is a tilt angle of 25 degrees, and mode five is a tilt angle of 30 degrees.

Still referring to FIG. 4, if the reset button 22 is not pushed again within ten seconds 56, in order to cycle through the modes that are available, then the device 10 exits mode setup 58. A user, may, however, cycle through the modes incrementally by pushing the reset button 22 during the ten second period 60.

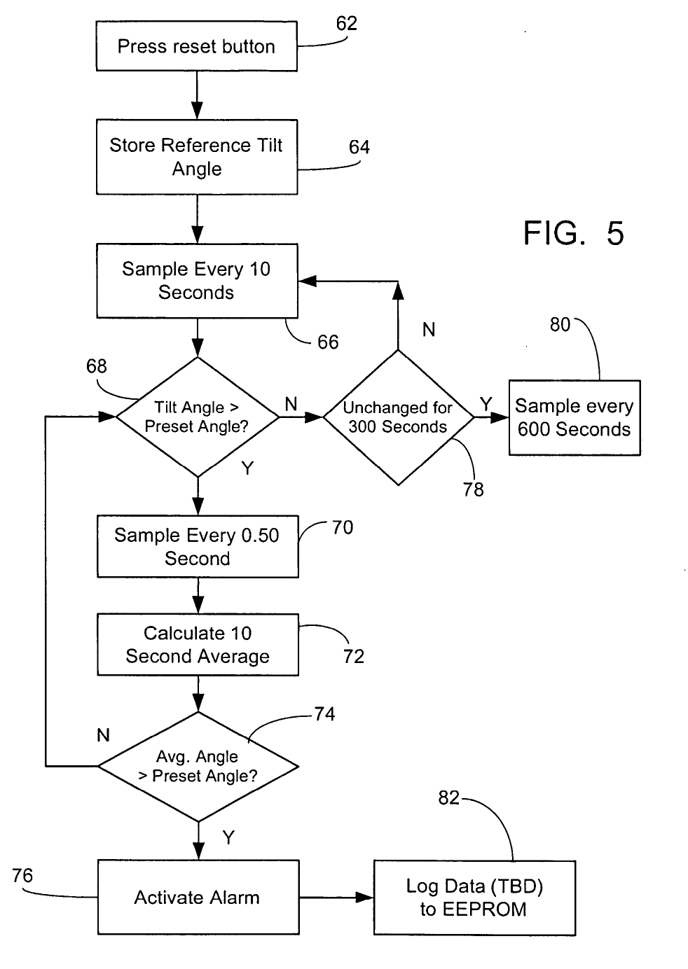

Referring now to FIG. 5, there is shown a flow chart providing the steps for monitoring the posture of a user. After selecting the operation mode, as previously described in FIG. 4, a user presses the reset button 22 in order to establish an initial reference tilt angle 62. From that point in time when the initial reference tilt angle is stored 64, the device 10 samples its angle every ten seconds 66. If the user's posture tile angle is greater than the selected allowable tilt angle 68, then the frequency of sampling is increased so that sampling occurs every 0.5 second 70. An average angle is then calculated for each ten second period 72. If the average angle is greater than the selected tilt angle 74, then the user is notified 76 by the alarm 18. The alarm 18 continues to notify the user of the violation of the tilt angle until the ten second average angle is less than the selected tilt angle.

Still referring to FIG. 5, when the user's posture tilt angle is not greater than the selected tilt angle, then sampling continues every ten seconds. However, in the embodiment shown in this figure, when the user's posture tilt angle has been the same for a period of five minutes (300 seconds) 78, then the device 10 will conserve battery power by reducing the frequency of sampling to five minute intervals 80. Essentially, if the same tilt angle has been measured for a five minute period, then it is highly probably that the user has taken it off, is sleeping, or the like. Thus, battery power is conserved during such activities. When sampling detects a different tilt angle, or when the reset button 22 is pressed, the frequency of sampling is increased to one sample per ten seconds.

All references, publications, and patents disclosed herein are expressly incorporated by reference.

Thus, it is seen that the apparatus and method of the present invention readily achieves the ends and advantages mentioned as well as those inherent therein. While certain preferred embodiments of the invention have been illustrated and described for the purpose of disclosure, numerous changes in the arrangement and construction of parts may be made by those skilled in the art, which changes are encompassed in the scope and spirit of the present invention as defined by the following claims.

What is claimed is:

1. A posture monitoring system, comprising:
a housing;
a timing sensor attached to the housing;
an angle detecting sensor attached to the timing sensor;
an alarm attached to the angle detecting circuit; and
a monitoring circuit attached to the angle detecting circuit for monitoring the maximum swings of the spine angle.

2. The system of claim 1, further comprising a recorder for recording the maximum swings of the spine angle.

3. The system of claim 1, wherein the recorder further comprises a memory storage device recorder for recording and playing back the recorded angles.

4. The system of claim 1, wherein the angle detecting circuit further comprises a digital angle detecting sensor.

5. The system of claim 1, wherein the alarm further comprises a light.

6. The system of claim 1, wherein the alarm further comprises a vibrator unit.

7. The system of claim 1, further comprising a switch attached to the housing, wherein the switch resets the angle detecting sensor.

8. A posture alert device for alerting a user of their posture, comprising:
a housing, wherein the housing has a length of up to 1.35 inches and a width of up to 1.10 inches;
a circuit board attached to the housing for setting the ranges of the posture alert device;
a tilt sensor attached to and operationally connected to the circuit board; and
a vibrator motor attached to and operationally connected to the circuit board.

9. The device of claim 8, further comprising a switch attached to the circuit board.

10. The device of claim 9, further comprising an attachment loop attached to the housing.

11. The device of claim 9, further comprising a clip attached to the housing.

12. A posture alert device for alerting a user of their posture, comprising:
a housing;
a first circuit board attached to the housing;
a second circuit board attached to the first circuit board, wherein the second circuit board is attached to the first circuit board at about a right angle;
a tilt sensor attached to the second circuit board; and
an alarm attached to the first circuit board.

13. The device of claim 12, further comprising a switch attached to the first circuit board, wherein the switch is operationally connected to the tilt sensor so that the switch resets the tilt sensor.

14. The device of claim 13, further comprising a clip attached to the housing so that the device attaches to a user.

15. The device of claim 14, wherein the housing has a thickness of from about 0.010 inch to about 0.070 inch.

16. The device of claim 12, wherein the alarm further comprises a vibrator unit.

17. The device of claim 12, wherein the alarm further comprises a light.

18. The device of claim 12, wherein the alarm further comprises a speaker.
